(19) United States
(12) Patent Application Publication
(10) Pub. No.: US 2019/0234959 A1
(43) Pub. Date: Aug. 1, 2019

(54) SYSTEM AND METHOD FOR CHARACTERIZING DRUG PRODUCT IMPURITIES

(71) Applicant: Regeneron Pharmaceuticals, Inc., Tarrytown, NY (US)

(72) Inventor: Shunhai Wang, Tarrytown, NY (US)

(21) Appl. No.: 16/259,095

(22) Filed: Jan. 28, 2019

Related U.S. Application Data
(60) Provisional application No. 62/624,366, filed on Jan. 31, 2018.

(51) Int. Cl. G01N 33/68; C07K 16/06; C12N 5/12; C12N 15/85; B01D 15/36; C07K 16/28; B01D 15/34

(52) U.S. Cl. CPC G01N 33/6848; C07K 16/065; C12N 5/12; C12N 15/85; C07K 2317/55; B01D 15/363; C07K 16/28; B01D 15/34; C12N 2015/8518; B01D 15/362

(57) ABSTRACT

Systems and methods for characterizing size and charge variant protein drug product impurities are provided.

### CROSS-REFERENCE TO RELATED APPLICATIONS

This application claims priority to U.S. Patent Provisional Patent Application No. 62/624,366 filed on Jan. 31, 2018, which is incorporated herein by reference.

### FIELD OF THE INVENTION

The invention is generally directed to protein separation methods and cell culture methods.

### BACKGROUND OF THE INVENTION

Monoclonal antibodies (mAbs) have been successfully employed to target a wide range of therapeutic areas over the last two decades (Walsh G., Biopharmaceutical benchmarks 2014, Nature biotechnology 2014; 32:992-1000; Lawrence S. Billion dollar babies—biotech drugs as blockbusters. Nature biotechnology 2007; 25:380-2).

Heterogeneity of antibodies is known in the art. For example, low molecular weight (LMW) species and high molecular weight (HMW) species are both examples of product-related impurities that contribute to the size heterogeneity of mAb products. The formation of HMW species within a therapeutic mAb drug product as a result of protein aggregation can potentially compromise both drug efficacy and safety. Proteolytic fragments may also contribute to the impurity profile of a product.

While mAbs possess a conserved covalent heterotetrameric structure consisting of two disulfide-linked heavy chains, each covalently linked through a disulfide bond to a light chain, these proteins often contain low levels of product-related impurities even after extensive purification steps. Low molecular weight (LMW) species (e.g., Fab fragments and monomer without an Fab arm) and high molecular weight (HMW) species (e.g., mAb trimer and mAb dimer) are both examples of product-related impurities that contribute to the size heterogeneity of mAb products. The formation of HMW species within a therapeutic mAb drug product as a result of protein aggregation can potentially compromise both drug efficacy and safety (e.g., eliciting unwanted immunogenic response) (Rosenberg A S. Effects of protein aggregates: an immunologic perspective. The AAPS journal 2006; 8:E501-7; Moussa E M, Panchal J P, Moorthy B S, Blum J S, Joubert M K, Narhi L O, et al. Immunogenicity of Therapeutic Protein Aggregates. Journal of Pharmaceutical Sciences 2016; 105:417-30). LMW species of any therapeutic protein may result from host cell protease activity during production. LMW species often have low or substantially reduced activity relative to the monomeric form of the antibody, while exposing novel epitopes that can lead to immunogenicity or potentially impact pharmacokinetic properties in vivo (Vlasak J, Ionescu R. Fragmentation of monoclonal antibodies. mAbs 2011; 3:253-63). As a result, both HMW and LMW species are considered critical quality attributes that are routinely monitored during drug development and as part of release testing of purified drug substance during manufacturing.

Molecular weight heterogeneity of mAb products is traditionally characterized by multiple orthogonal analytical methods (Michels D A, Parker M, Salas-Solano O. Electrophoresis 2012; 33:815-26). One of the most commonly used techniques to assess mAb product purity is SDS-PAGE, performed under non-reducing conditions. During analysis, minor bands corresponding to LMW species can be routinely observed and quantified, including H2L (2 heavy chains and 1 light chain), H2 (2 heavy chains), HL (1 heavy chain and 1 light chain), HC (1 heavy chain), and LC (1 light chain) species, with respect to antibodies (Liu H, Gaza-Bulseco G, Chumsae C, Newby-Kew A. Biotechnology Letters 2007; 29:1611-22).

Proteolytic fragments may also be observed. The proposed identity of each minor band can be supported by N-terminal sequencing via Edman degradation, in-gel tryptic digestion followed by mass spectrometry analysis, and western blot analysis using anti-Fc and anti-light chain antibodies. However, any proposed structures resulting from these methods cannot be unambiguously confirmed at the intact protein level. Furthermore, sample preparation conditions employed in SDS-PAGE experiments can generate LMW artifacts through disulfide bond scrambling, which can lead to overestimations of minor LMW species (Zhu Z C, et al. Journal of Pharmaceutical and Biomedical Analysis, 83:89-95 (2013)).

More recently, capillary electrophoresis-sodium dodecyl sulfate (CE-SDS) has emerged as a modern equivalent of SDS-PAGE, offering superior reproducibility, sensitivity, and throughput (Rustandi R R, Washabaugh M W, Wang Y. Electrophoresis, 29:3612-20 (2013); Lacher N A, et al., Journal of Separation Science, 33:218-27 (2010); Hunt G, et al., Journal of Chromatography A 744:295-301 (1996)). During CE-SDS analysis of mAb products, minor peaks with shorter migration times (LMW forms) than the intact antibody can be routinely observed. Unlike SDS-PAGE analysis, these LMW impurities cannot be extracted or subjected to further analyses. As a result, the identities of LMW impurities observed in CE-SDS methods are often proposed solely based on empirical knowledge.

Accurate mass measurement of intact mAb proteins by modern mass spectrometers has become increasingly popular in the biopharmaceutical industry as one of the most reliable identification techniques (Kaltashov I A, et al., Journal of the American Society for Mass Spectrometry, 21:323-37 (2010); Zhang H, Cui W, Gross M L. FEBS Letters, 588:308-17 (2014)). Specifically, a variety of “hyphenated chromatography-mass spectrometry” methods have demonstrated the capability of detecting low-abundance impurities in mAb products and providing highly detailed analyses that cannot be achieved by either SDS-PAGE or CE-SDS methods (ie J C, Bondarenko P V. Journal of the American Society for Mass Spectrometry; 16:307-11 (2015); Haberger M, et al. mAbs 8:331-9 (2016)). For example, reversed-phase chromatography (RPLC) coupled to mass spectrometry can be used to detect free light chain and associated post-translational modifications (e.g. cysteinylation and glutathionylation) present in mAb drug products. However, compared to SDS-PAGE and CE-SDS methods, RPLC often lacks sufficient resolution to separate LMW species and thus fails to elucidate the complete LMW profile. For example, the identification of H2L species in mAb drug products has never been reported by RPLC-based intact mass analysis, owing to its low abundance and poor resolution from the main intact antibody.

Another MS-based technique that is promising for characterizing mAb product-related impurities is native electrospray ionization mass spectrometry (Native ESI-MS), which is particularly informative when coupled with size exclusion chromatography (SECX Haberger M, et al. mAbs; 8:331-339 (2016)). However, the LMW species identified in native SEC-MS analysis are often not the same as those identified by SDS-PAGE or CE-SDS, due to significantly different experimental conditions used between methods. Specifically, the sample preparation required for SDS-PAGE and CE-SDS often starts with protein denaturation, where the non-covalent interactions between the N-terminal regions of HC-LC pairs and the C-terminal regions of the HC-HC pairs are disrupted. As a result, LMW impurities such as H2L, half antibody, and free light chain species are able to dissociate from the mAb molecule if the interchain disulfide bonds are broken.

In comparison, native SEC-MS analyzes the mAb samples under near native conditions, permitting the strong non-covalent interchain interactions to be preserved and allowing the four-chain structure of the mAb molecule to be maintained even if the interchain disulfide bonds are broken. Although advances in SEC column chemistry have made it possible to use denaturing buffers (e.g. 30/o acetonitrile, 0.1% FA and 0.1% TFA) that are normally used in reversed-phase chromatography for SEC separation and direct coupling to online mass spectrometry analysis (Liu H, Gaza-Bulseco G, Chumsae C. Journal of the American Society for Mass Spectrometry, 20:2258-64 (2009), the LC resolution is still sub-optimal to detect many LMW species.

It is an object of the invention to provide systems and methods for the characterization of size variants of protein drug impurities.

It is another object of the invention to provide protein drug products with reduced levels of impurities.

It is still another object of the invention to provide methods of producing protein drug products with reduced protein drug product impurities.

### SUMMARY OF THE INVENTION

Systems and methods for characterizing size and charge variant protein drug product impurities are provided. One embodiment uses size exclusion chromatography (SEC) with an aqueous mobile phase coupled with native mass spectrometry analysis to detect and characterize size variant protein drug product impurities. Another embodiment uses ion exchange chromatography (IEX), preferably strong cation exchange chromatography with an aqueous mobile phase coupled with native mass spectrometry analysis to characterize protein drug product impurities. In one embodiment, after removal of the N-linked glycans from the protein drug product, for example an antibody drug product, the elution of size or charge variant impurities from the SEC or IEX column respectively is determined by the size and/or charge of the molecular weight species.

The disclosed systems and methods can be used to characterize size variants, charge variants, antibody-antigen binding, post-translational modification (PTM) characterizations, characterization of partially reduced and alkylated mAb, dimer characterization for co-formulated drugs, IgG4 Fab exchange characterization, and highly heterogeneous sample characterization using charge reduction. Exemplary PTMs that can be detected and identified that contribute to acidic variants include but are not limited to glycation, glucuronylation, carboxymethylation, sialylation, non-consensus glycosylation at Fab region. PTMs that can be detected and identified that contribute to basic variants include but are not limited to succinimide formation, N-terminal glutamine (not converted to pyroglutamate), C-terminal Lys and non-/partial-glycosylated species.

Exemplary low molecular weight (LMW) protein drug product impurities that can be detected and characterized with the disclosed systems include but are not limited to precursors, degradation products, truncated species, proteolytic fragments including Fab, ligand or receptor fragments or heavy chain fragments, free light chain, half antibody, H2L, H2, HL, HC, or combinations thereof.

Exemplary high molecular weight (HMW) impurities include but are not limited to mAb trimers and mAb dimers.

Exemplary intermediate HMW include but are not limited to monomer with extra light chains (H2L3 and H2L4 species), monomer plus Fab fragments complexes, Fab2-Fab2, Fc-Fc, and Fab2-Fc.

The disclosed SEC-native MS and IEX-native MS systems and methods provide detailed variant protein drug product identification information. The reliable identification and detailed structural information obtained with the disclosed systems and methods is highly valuable for in-depth characterization of impurities in protein drug products, which is often required for late-stage molecule development. Furthermore, because the disclosed systems and methods use gentler sample preparations than either SDS-PAGE or CE-SDS does, it is less likely to generate artifacts. The disclosed systems and methods can be used as a semi-quantitative analysis to compare the impurity profiles between samples or simply applied qualitatively.

One embodiment provides a protein drug product containing a protein drug and an excipient, wherein the protein drug product comprises between 0.05 and 30.0% w/w of low molecular weight, high molecular weight, intermediate high molecular weight protein drug impurities, or combinations thereof.

A preferred embodiment provides a protein drug product containing a protein drug and an excipient, wherein the protein drug product comprises between 0.05 and 30.0% w/w of intermediate high molecular weight protein drug impurities

The protein drug product can be an antibody, a fusion protein, recombinant protein, or a combination thereof. In other embodiments, the drug product contains between 1 to 25%, 1 to 15%, 1 to 10%, or 1 to 5% w/w of intermediate high molecular weight protein drug impurities.

Another embodiment provides a method for characterizing size or charge variant protein drug product impurities including the steps of deglycosylating a protein drug product sample, separating protein components of the protein drug product sample by SEC or IEX chromatography, and analyzing the separated protein components by native mass spectrometry to characterize the size or charge variant protein drug product impurities in the protein drug product sample. The method further provides an optional reducing step. The protein drug product sample can be taken from a fed-batch culture. As noted above, the protein drug product can be an antibody, a fusion protein, recombinant protein, or a combination thereof.

Still another embodiment provides a method of producing an antibody, including the steps of culturing cells producing the antibody in a cell culture, obtaining a sample from the cell culture, characterizing and quantifying size, or charge variant protein drug impurities in the sample according to the methods described above and modifying one or more culture conditions of the cell culture to reduce the amount of characterized low molecular protein drug impurities produced during cell culture of the antibody. In some embodiments, the sample is taken during the cell culture at any interval. In other embodiments, the sample is taken following production culture, following protein harvest or following purification. The one or more conditions of the cell culture that are changed to reduce the amount of low molecular weight protein drug impurities can be selected from the group consisting of temperature, pH, cell density, amino acid concentration, osmolality, growth factor concentration, agitation, gas partial pressure, surfactants, or combinations thereof. The cells can be eukaryotic or prokaryotic. The cells can be Chinese Hamster Ovary (CHO) cells (e.g. CHO K1, DXB-11 CHO, Veggie-CHO), COS cells (e.g. COS-7), retinal cells, Vero cells, CV1 cells, kidney cells (e.g. HEK293, 293 EBNA, MSR 293, MDCK, HaK, BHK21), HeLa cells, HepG2 cells, W138 cells, MRC 5 cells, Colo25 cells, HB 8065 cells. HL-60 cells, lymphocyte cells, e.g. autologous T cells, Jurkat (T lymphocytes) or Daudi (B lymphocytes), A431 (epidermal) cells, U937 cells, 3T3 cells, L cells, C127 cells, SP2/0 cells, NS-0 cells, MMT cells, stem cells, tumor cells, and a cell line derived from any of the aforementioned cells. In one embodiment the cells are hybridoma or quadroma cells. Still another embodiment provides an antibody produced by the methods described herein.

Yet another embodiment provides a system for characterizing size and charge variant drug impurities. The system includes an SEC or IEX chromatography system linked to an aqueous mobile phase and in fluid communication with a native mass spectrometry system.

### BRIEF DESCRIPTION OF THE DRAWINGS

FIGS. 1A and 1B are chromatograms of Online Native SEC-MS separation of mAb-1 drug substance sample. FIG. 1A is the ultraviolet profile and FIGS. 1B-1E is the mass spectrometry profile of monomer, dimer, trimer, and quatromer, respectively.

FIG. 2A is a mass spectrometry profile of Fab₂ homodimer from the mAb-1 drug substance sample. FIG. 2B is the mass spectrometry profile of Fab2-Fc heterodimer from the mAb-1 drug substance sample. FIG. 2C is the mass spectrometry profile of an Fc homodimer from the mAb-1 drug substance sample. FIG. 2D is total ion chromatograph of the separation of mAb-1.

FIG. 3A shows a total ion chromatogram of Online Native SEC-MS separation of mAb-2 drug substance sample. FIG. 3B shows the mass spectrometry profile of low molecular weight from the fraction centered at 26 min. FIG. 3C shows the mass spectrometry profile of low molecular weight from the fraction centered at 31 min.

FIG. 4 is a total ion current chromatogram of Online Native SEC-MS of mAb-1 drug substance from an enriched LMW sample (deglycosylated).

FIG. 5A is a total ion current chromatogram of Online Native SEC-MS of mAb-3 drug substance showing detection of dimer, intermediate HMW, and monomer impurities. FIG. 5B is a total ion current chromatogram showing detection of monomer impurities. FIGS. 5C-5E are mass spectrometry profiles of dimer, intermediate HMW, and monomer impurities.

FIG. 6 is the deconvoluted mass spectra of the intermediate HMW species in mAb-3 showing the predict mass of H2L3 as 167,850 Da.

FIG. 7A shows extracted ion chromatographs of mAb-4 showing detection of charge variant impurities. FIG. 7B shows the mass spectrometry profile of the indicated charge variant impurities.

FIG. 8 is a total ion chromatogram of mAb-4 showing characterization of charge variants at the subdomain level by native SCX-MS.

FIG. 9A shows extracted ion chromatograms of Fab2 fragments characterized by native SCX-MS. FIG. 9B shows mass spectrometry profiles of charge variants.

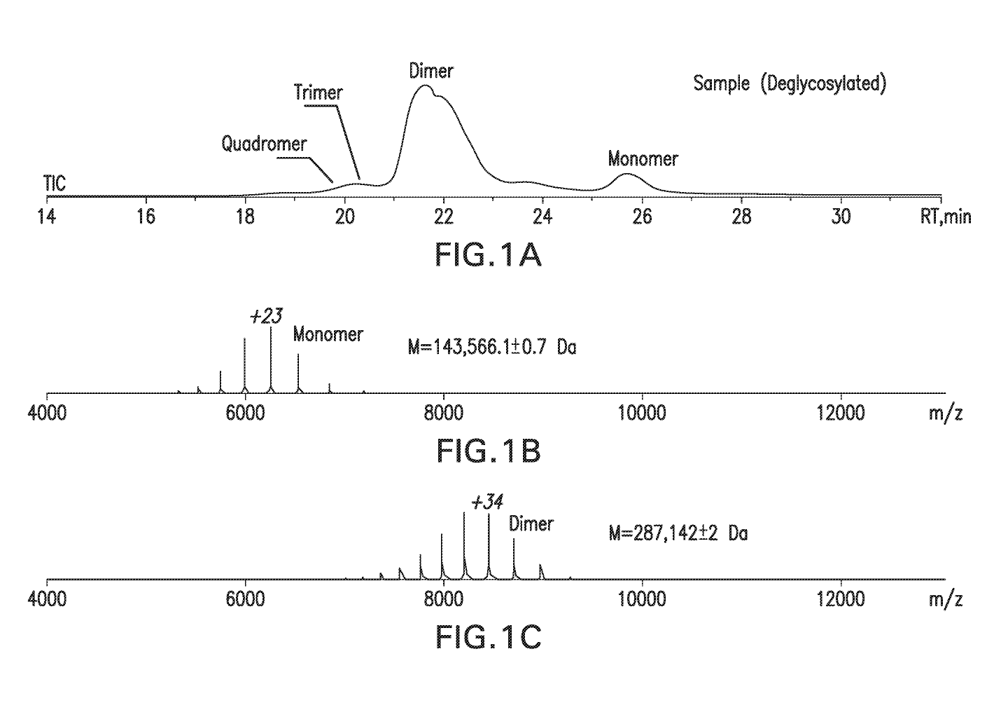

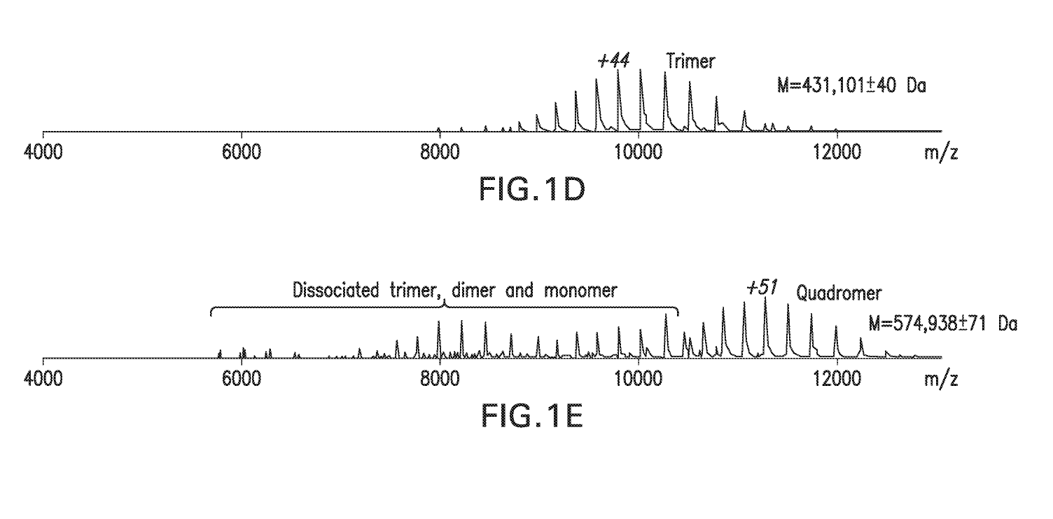

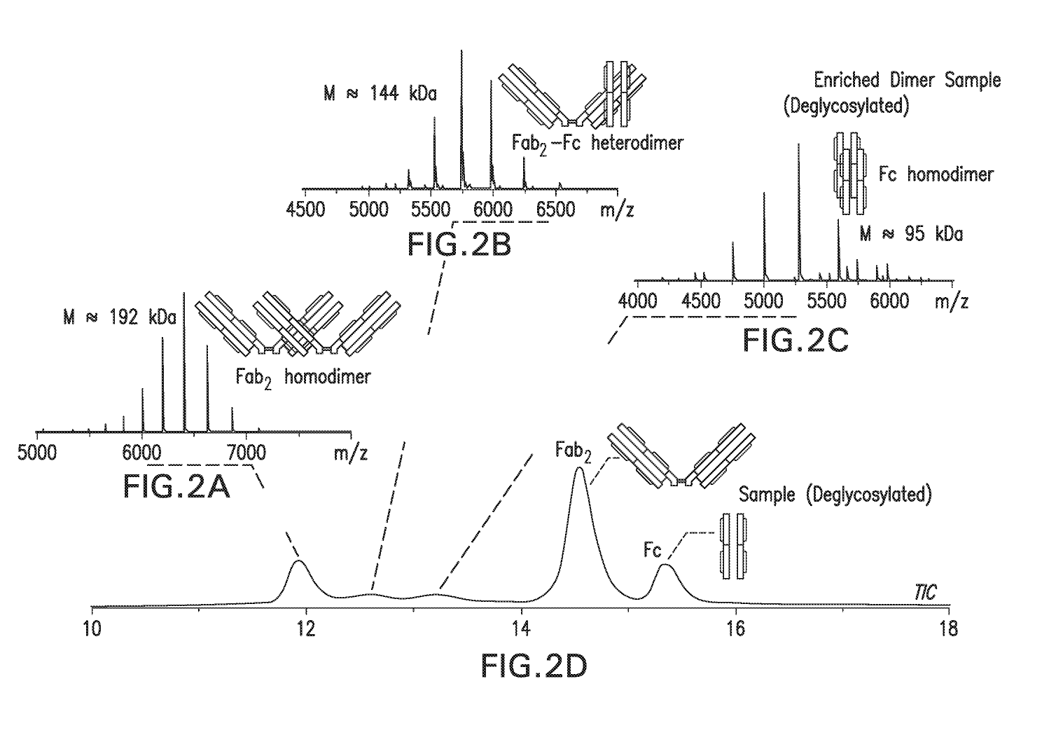

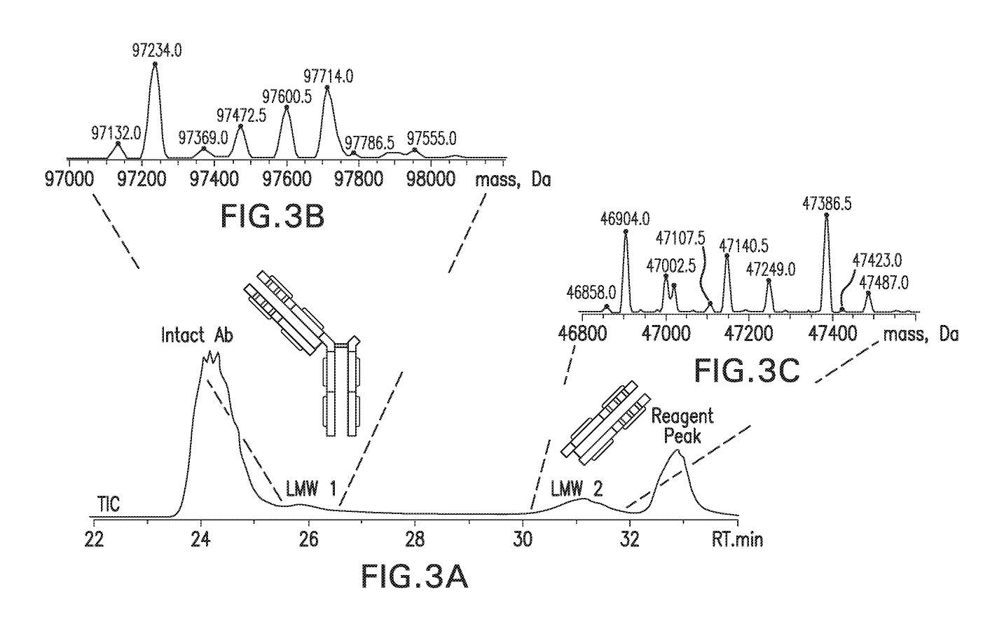

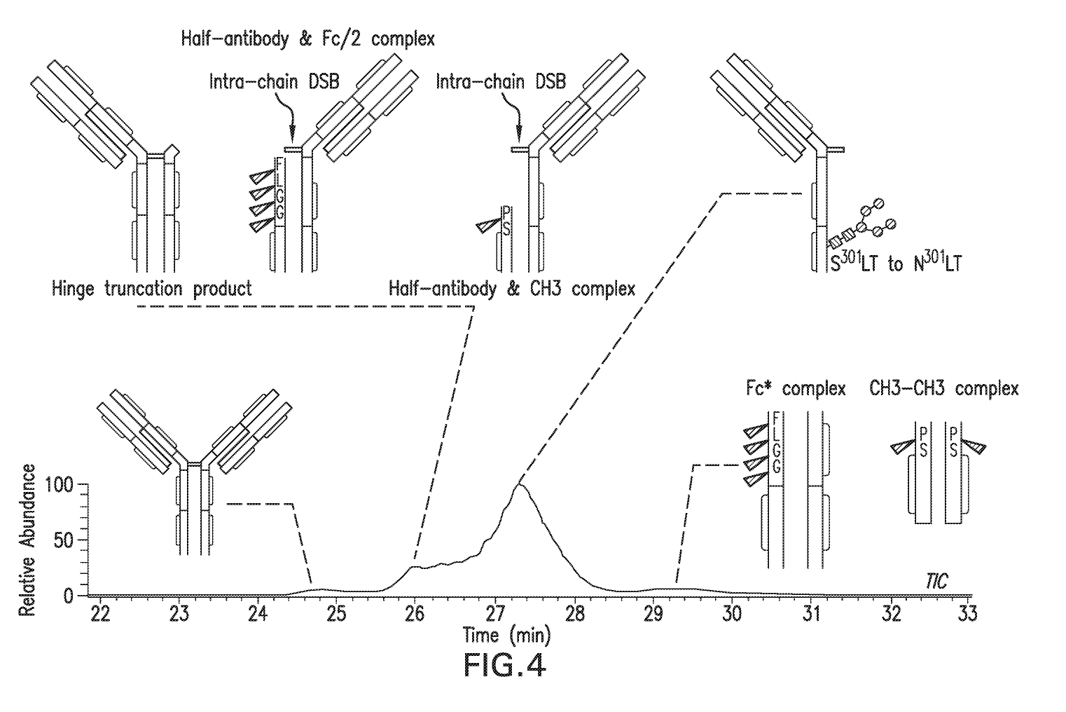

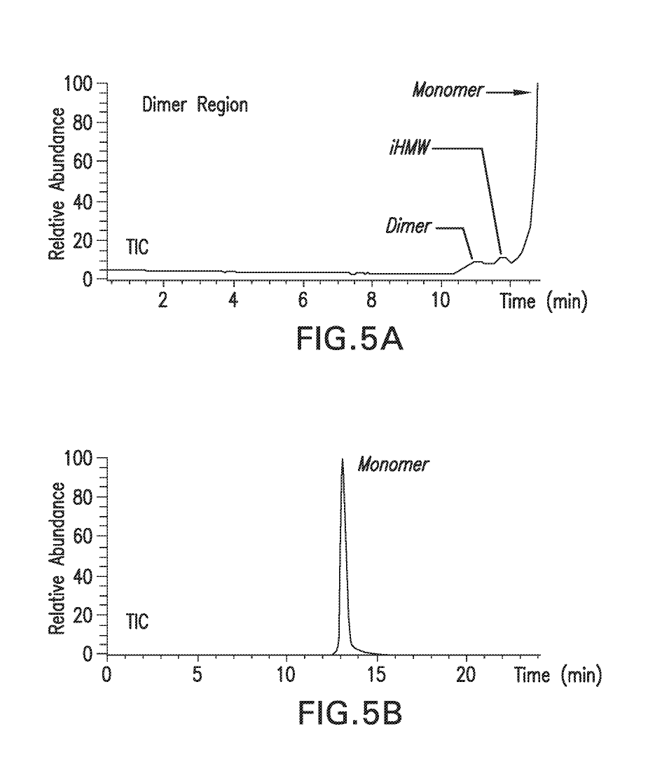

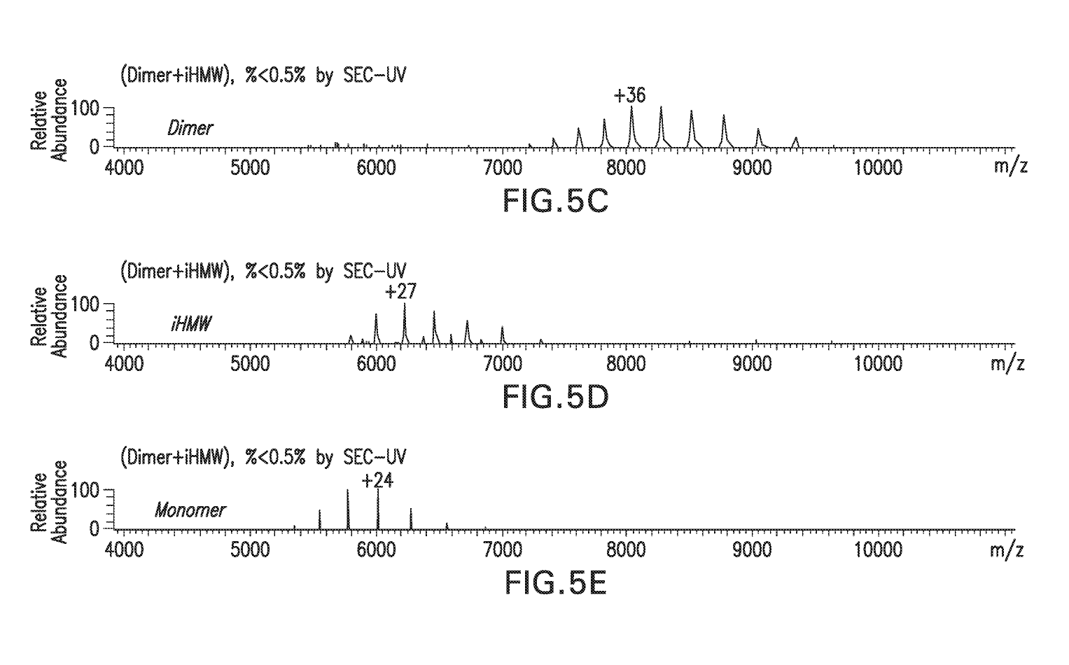

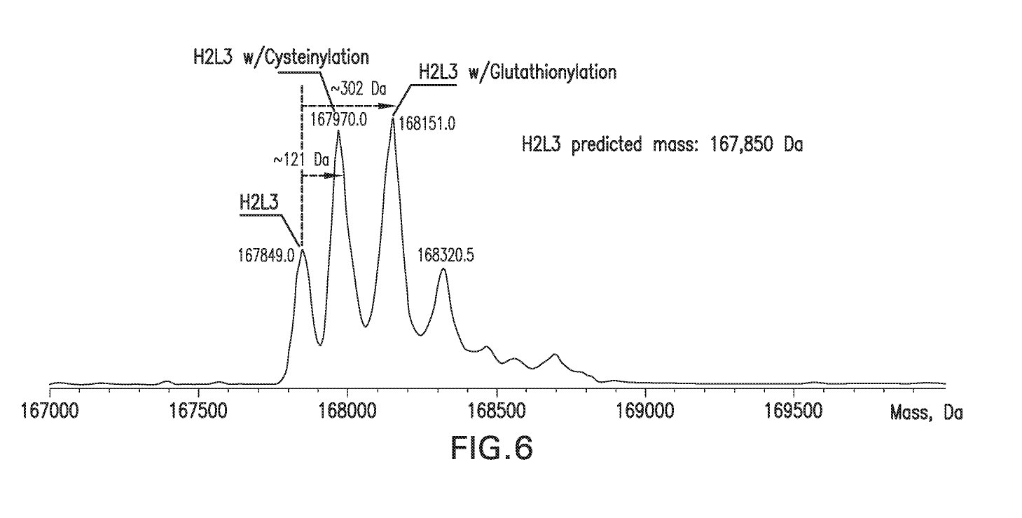

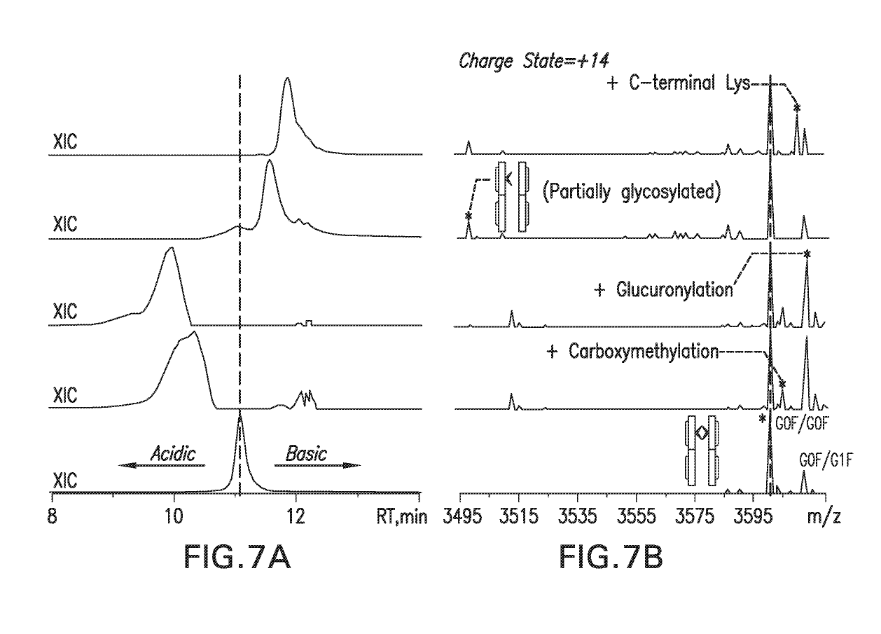

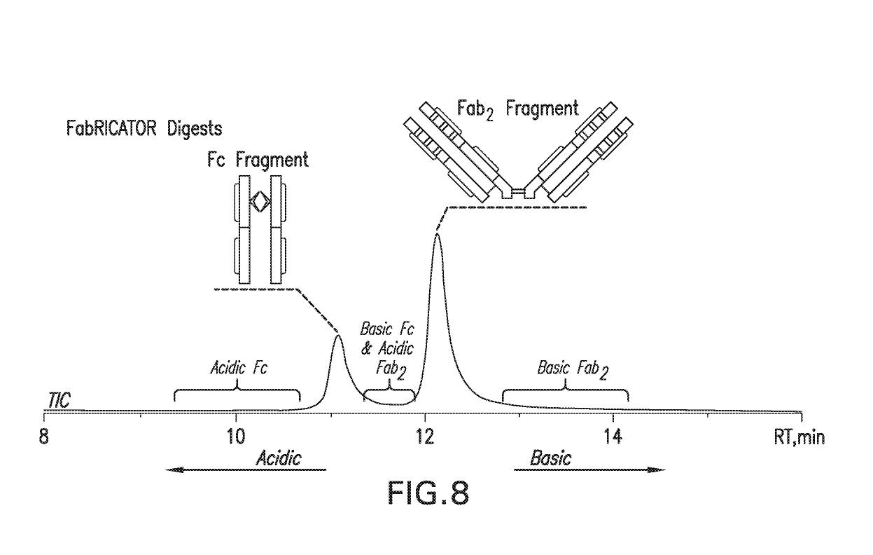

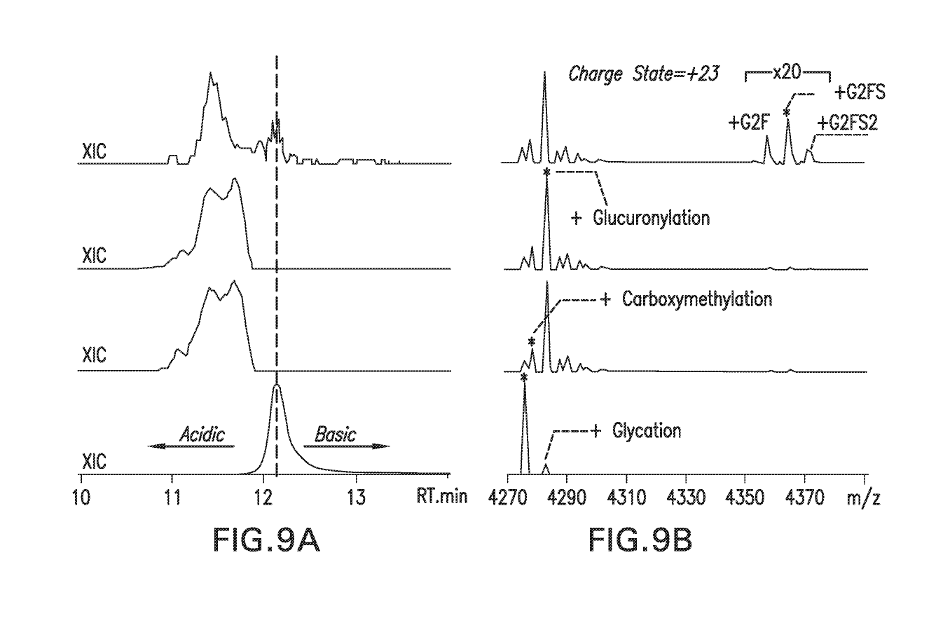

### DETAILED DESCRIPTION OF THE INVENTION

### I. Definitions

The use of the terms “a,” “an,” “the,” and similar referents in the context of describing the presently claimed invention (especially in the context of the claims) are to be construed to cover both the singular and the plural, unless otherwise indicated herein or clearly contradicted by context.

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/−10%; in other embodiments the values may range in value either above or below the stated value in a range of approx. +/−5%; in other embodiments the values may range in value either above or below the stated value in a range of approx. +/−2%; in other embodiments the values may range in value either above or below the stated value in a range of approx. +/−1%. The preceding ranges are intended to be made clear by context, and no further limitation is implied. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

The term “low molecular weight (LMW) protein drug impurity” includes but is not limited to precursors, degradation products, truncated species, proteolytic fragments including Fab fragments, Fc or heavy chain fragments, ligand or receptor fragments, H2L (2 heavy chains and 1 light chain), H2 (2 heavy chains), HL (1 heavy chain and 1 light chain), HC (1 heavy chain), and LC (1 light chain) species. A LMW protein drug impurity can be any variant which is an incomplete version of the protein product, such as one or more components of a multimeric protein. Protein drug impurity, drug impurity or product impurity are terms that may be used interchangeably throughout the specification. LMW drug or product impurities are generally considered molecular variants with properties such as activity, efficacy, and safety that may be different from those of the desired drug product.

Degradation of protein product is problematic during production of the protein drug product in cell culture systems. For example, proteolysis of a protein product may occur due to release of proteases in cell culture medium. Medium additives, such as soluble iron sources added to inhibit metalloproteases, or serine and cysteine proteases inhibitors, have been implemented in cell culture to prevent degradation (Clincke, M.-F., et al, BMC Proc. 2011, 5, P115). C-terminal fragments may be cleaved during production due to carboxyl peptidases in the cell culture (Dick, L W et al, Biotechnol Bioeng 2008; 100:1132-43).

The term “high molecular weight (HMW) protein drug impurity” includes but is not limited to mAb trimers and mAb dimers. HMW species can be divided into two groups: 1) monomer with extra light chains (H2L3 and H2L4 species) and 2) monomer plus Fab fragments complexes. In addition, after treatment with IdeS enzymatic digestion, different dimerized fragments (Fab2-Fab2, Fc-Fc and Fab2-Fc) are formed.

“Protein” refers to a molecule comprising two or more amino acid residues joined to each other by a peptide bond. Protein includes polypeptides and peptides and may also include modifications such as glycosylation, lipid attachment, sulfation, gamma-carboxylation of glutamic acid residues, alkylation, hydroxylation and ADP-ribosylation. Proteins can be of scientific or commercial interest, including protein-based drugs, and proteins include, among other things, enzymes, ligands, receptors, antibodies and chimeric or fusion proteins. Proteins are produced by various types of recombinant cells using well-known cell culture methods and are generally introduced into the cell by genetic engineering techniques (e.g., such as a sequence encoding a chimeric protein, or a codon-optimized sequence, an intronless sequence, etc.) where it may reside as an episome or be integrated into the genome of the cell.

“Antibody” refers to an immunoglobulin molecule consisting of four polypeptide chains, two heavy (H) chains and two light (L) chains inter-connected by disulfide bonds. Each heavy chain has a heavy chain variable region (HCVR or VH) and a heavy chain constant region. The heavy chain constant region contains three domains, CH1, CH2 and CH3. Each light chain has a light chain variable region and a light chain constant region. The light chain constant region consists of one domain (CL). The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. The term “antibody” includes reference to both glycosylated and non-glycosylated immunoglobulins of any isotype or subclass. The term “antibody” includes antibody molecules prepared, expressed, created or isolated by recombinant means, such as antibodies isolated from a host cell transfected to express the antibody. The term antibody also includes bispecific antibody, which includes a heterotetrameric immunoglobulin that can bind to more than one different epitope. Bispecific antibodies are generally described in US Patent Application Publication No. 2010/0331527, which is incorporated by reference into this application.

“Fc fusion proteins” comprise part or all of two or more proteins, one of which is an Fc portion of an immunoglobulin molecule, which are not otherwise found together in nature. Preparation of fusion proteins comprising certain heterologous polypeptides fused to various portions of antibody-derived polypeptides (including the Fc domain) has been described, e.g., by Ashkenazi et al., Proc. Natl. Acad. Sci USA 88: 10535, 1991; Byrn et al., Nature 344:677, 1990; and Hollenbaugh et al., “Construction of Immunoglobulin Fusion Proteins”, in Current Protocols in Immunology, Suppl. 4, pages 10.19.1-10.19.11, 1992. “Receptor Fc fusion proteins” comprise one or more extracellular domain(s) of a receptor coupled to an Fc moiety, which in some embodiments comprises a hinge region followed by a CH2 and CH3 domain of an immunoglobulin. In some embodiments, the Fc-fusion protein comprises two or more distinct receptor chains that bind to a one or more ligand(s). For example, an Fc-fusion protein is a trap, such as for example an IL-1 trap or VEGF trap.

“Cell culture” refers to the propagation or proliferation of cells in a vessel, such as a flask or bioreactor, and includes but is not limited to fed-batch culture, continuous culture, perfusion culture and the like.

### II. Protein Drug Products

A. Proteins of Interest

A protein drug product can be any protein of interest suitable for expression in prokaryotic or eukaryotic cells and can be used in engineered host cell. For example, the protein of interest includes, but is not limited to, an antibody or antigen-binding fragment thereof, a chimeric antibody or antigen-binding fragment thereof, an ScFv or fragment thereof, an Fc-fusion protein or fragment thereof, a growth factor or a fragment thereof, a cytokine or a fragment thereof, or an extracellular domain of a cell surface receptor or a fragment thereof. Proteins of interest may be simple polypeptides consisting of a single subunit, or complex multisubunit proteins comprising two or more subunits. The protein of interest may be a biopharmaceutical product, food additive or preservative, or any protein product subject to purification and quality standards.

In some embodiments, the protein product (protein of interest) is an antibody, a human antibody, a humanized antibody, a chimeric antibody, a monoclonal antibody, a multispecific antibody, a bispecific antibody, an antigen binding antibody fragment, a single chain antibody, a diabody, triabody or tetrabody, a Fab fragment or a F(ab′)2 fragment, an IgD antibody, an IgE antibody, an IgM antibody, an IgG antibody, an IgG1 antibody, an IgG2 antibody, an IgG3 antibody, or an IgG4 antibody. In one embodiment, the antibody is an IgG1 antibody. In one embodiment, the antibody is an IgG2 antibody. In one embodiment, the antibody is an IgG4 antibody. In one embodiment, the antibody is a chimeric IgG2/IgG4 antibody. In one embodiment, the antibody is a chimeric IgG2/IgG1 antibody. In one embodiment, the antibody is a chimeric IgG2/IgG1/IgG4 antibody.

In some embodiments, the antibody is selected from the group consisting of an anti-Programmed Cell Death 1 antibody (e.g. an anti-PDI antibody as described in U.S. Pat. Appln. Pub. No. US2015/0203579A1), an anti-Programmed Cell Death Ligand-1 (e.g. an anti-PD-L1 antibody as described in in U.S. Pat. Appln. Pub. No. US2015/0203580A1), an anti-D114 antibody, an anti-Angiopoetin-2 antibody (e.g. an anti-ANG2 antibody as described in U.S. Pat. No. 9,402,898), an anti-Angiopoetin-Like 3 antibody (e.g. an anti-AngPtl3 antibody as described in U.S. Pat. No. 9,018,356), an anti-platelet derived growth factor receptor antibody (e.g. an anti-PDGFR antibody as described in U.S. Pat. No. 9,265,827), an anti-Erb3 antibody, an anti-Prolactin Receptor antibody (e.g. anti-PRLR antibody as described in U.S. Pat. No. 9,302,015), an anti-Complement 5 antibody (e.g. an anti-C5 antibody as described in U.S. Pat. Appln. Pub. No US2015/0313194A1), an anti-TNF antibody, an anti-epidermal growth factor receptor antibody (e.g. an anti-EGFR antibody as described in U.S. Pat. No. 9,132,192 or an anti-EGFRvIII antibody as described in U.S. Pat. Appln. Pub. No. US2015/0259423A1), an anti-Proprotein Convertase Subtilisin Kexin-9 antibody (e.g. an anti-PCSK9 antibody as described in U.S. Pat. No. 8,062,640 or U.S. Pat. Appln. Pub. No. US2014/0044730A1), an anti-Growth And Differentiation Factor-8 antibody (e.g. an anti-GDF8 antibody, also known as anti-myostatin antibody, as described in U.S. Pat. No. 8,871,209 or 9,260,515), an anti-Glucagon Receptor (e.g. anti-GCGR antibody as described in U.S. Pat. Appln. Pub. Nos. US2015/0337045A1 or US2016/0075778A1), an anti-VEGF antibody, an anti-IL1R antibody, an interleukin 4 receptor antibody (e.g an anti-IL4R antibody as described in U.S. Pat. Appln. Pub. No. US2014/0271681A1 or U.S. Pat. No. 8,735,095 or 8,945,559), an anti-interleukin 6 receptor antibody (e.g. an anti-IL6R antibody as described in U.S. Pat. Nos. 7,582,298, 8,043,617 or 9,173,880), an anti-IL1 antibody, an anti-1L2 antibody, an anti-1L3 antibody, an anti-1L4 antibody, an anti-IL5 antibody, an anti-IL6 antibody, an anti-IL7 antibody, an anti-interleukin 33 (e.g. anti-IL33 antibody as described in U.S. Pat. Appln. Pub. Nos. US2014/0271658A1 or US2014/0271642A1), an anti-Respiratory syncytial virus antibody (e.g. anti-RSV antibody as described in U.S. Pat. Appln. Pub. No. US2014/0271653A1), an anti-Cluster of differentiation 3 (e.g. an anti-CD3 antibody, as described in U.S. Pat. Appln. Pub. Nos. US2014/0088295A1 and US20150266966A1, and in U.S. Application No. 62/222,605), an anti-Cluster of differentiation 20 (e.g. an anti-CD20 antibody as described in U.S. Pat. Appln. Pub. Nos. US2014/0088295A1 and US20150266966A1, and in U.S. Pat. No. 7,879,984), an anti-CD19 antibody, an anti-CD28 antibody, an anti-Cluster of Differentiation-48 (e.g. anti-CD48 antibody as described in U.S. Pat. No. 9,228,014), an anti-Fel dl antibody (e.g. as described in U.S. Pat. No. 9,079,948), an anti-Middle East Respiratory Syndrome virus (e.g. an anti-MERS antibody as described in U.S. Pat. Appln. Pub. No. US2015/0337029A1), an anti-Ebola virus antibody (e.g. as described in U.S. Pat. Appln. Pub. No. US2016/0215040), an anti-Zika virus antibody, an anti-Lymphocyte Activation Gene 3 antibody (e.g. an anti-LAG3 antibody, or an anti-CD223 antibody), an anti-Nerve Growth Factor antibody (e.g. an anti-NGF antibody as described in U.S. Pat. Appln. Pub. No. US2016/0017029 and U.S. Pat. Nos. 8,309,088 and 9,353,176) and an anti-Activin A antibody. In some embodiments, the bispecific antibody is selected from the group consisting of an anti-CD3×anti-CD20 bispecific antibody (as described in U.S. Pat. Appln. Pub. Nos. US2014/0088295A1 and US20150266966A1), an anti-CD3×anti-Mucin 16 bispecific antibody (e.g., an anti-CD3×anti-Muc16 bispecific antibody), and an anti-CD3×anti-Prostate-specific membrane antigen bispecific antibody (e.g., an anti-CD3×anti-PSMA bispecific antibody). In some embodiments, the protein of interest is selected from the group consisting of abciximab, adalimumab, adalimumab-atto, ado-trastuzumab, alemtuzumab, alirocumab, atezolizumab, avelumab, basiliximab, belimumab, benralizumab, bevacizumab, bezlotoxumab, blinatumomab, brentuximab vedotin, brodalumab, canakinumab, capromab pendetide, certolizumab pegol, cemiplimab, cetuximab, denosumab, dinutuximab, dupilumab, durvalumab, eculizumab, elotuzumab, emicizumab-kxwh, emtansinealirocumab, evinacumab, evolocumab, fasinumab, golimumab, guselkumab, ibritumomab tiuxetan, idarucizumab, infliximab, infliximab-abda, infliximab-dyyb, ipilimumab, ixekizumab, mepolizumab, necitumumab, nesvacumab, nivolumab, obiltoxaximab, obinutuzumab, ocrelizumab, ofatumumab, olaratumab, omalizumab, panitumumab, pembrolizumab, pertuzumab, ramucirumab, ranibizumab, raxibacumab, reslizumab, rinucumab, rituximab, sarilumab, secukinumab, siltuximab, tocilizumab, tocilizumab, trastuzumab, trevogrumab, ustekinumab, and vedolizumab.

In some embodiments, the protein of interest is a recombinant protein that contains an Fc moiety and another domain, (e.g., an Fc-fusion protein). In some embodiments, an Fc-fusion protein is a receptor Fc-fusion protein, which contains one or more extracellular domain(s) of a receptor coupled to an Fc moiety. In some embodiments, the Fc moiety comprises a hinge region followed by a CH2 and CH3 domain of an IgG. In some embodiments, the receptor Fc-fusion protein contains two or more distinct receptor chains that bind to either a single ligand or multiple ligands. For example, an Fc-fusion protein is a TRAP protein, such as for example an IL-1 trap (e.g., rilonacept, which contains the IL-1RAcP ligand binding region fused to the I-1R1 extracellular region fused to Fc of hIgG1; see U.S. Pat. No. 6,927,004, which is herein incorporated by reference in its entirety), or a VEGF trap (e.g., aflibercept or ziv-aflibercept, which comprises the Ig domain 2 of the VEGF receptor Flt1 fused to the Ig domain 3 of the VEGF receptor Flk1 fused to Fc of hIgG1; see U.S. Pat. Nos. 7,087,411 and 7,279,159). In other embodiments, an Fc-fusion protein is a ScFv-Fc-fusion protein, which contains one or more antigen-binding domain(s), such as a variable heavy chain fragment and a variable light chain fragment, of an antibody coupled to an Fc moiety.

B. Cell Culture

The protein of interest can be produced in a “fed-batch cell culture” or “fed-batch culture” which refers to a batch culture wherein the cells and culture medium are supplied to the culturing vessel initially, and additional culture nutrients are slowly fed, in discrete increments, to the culture during culturing, with or without periodic cell and/or product harvest before termination of culture. Fed-batch culture includes “semi-continuous fed-batch culture” wherein periodically whole culture (which may include cells and medium) is removed and replaced by fresh medium. Fed-batch culture is distinguished from simple “batch culture” whereas all components for cell culturing (including the animal cells and all culture nutrients) are supplied to the culturing vessel at the start of the culturing process in batch culture. Fed-batch culture may be different from “perfusion culture” insofar as the supernatant is not removed from the culturing vessel during a standard fed-batch process, whereas in perfusion culturing, the cells are restrained in the culture by, e.g., filtration, and the culture medium is continuously or intermittently introduced and removed from the culturing vessel. However, removal of samples for testing purposes during fed-batch cell culture is contemplated. The fed-batch process continues until it is determined that maximum working volume and/or protein production is reached, and protein is subsequently harvested.

The protein of interest can be produced in a continuous cell culture. The phrase “continuous cell culture” relates to a technique used to grow cells continually, usually in a particular growth phase. For example, if a constant supply of cells is required, or the production of a particular protein of interest is required, the cell culture may require maintenance in a particular phase of growth. Thus, the conditions must be continually monitored and adjusted accordingly in order to maintain the cells in that particular phase.

The terms “cell culture medium” and “culture medium” refer to a nutrient solution used for growing mammalian cells that typically provides the necessary nutrients to enhance growth of the cells, such as a carbohydrate energy source, essential (e.g. phenylalanine, valine, threonine, tryptophan, methionine, leucine, isoleucine, lysine, and histidine) and nonessential (e.g. alanine, asparagine, aspartic acid, cysteine, glutamic acid, glutamine, glycine, proline, serine, and tyrosine) amino acids, trace elements, energy sources, lipids, vitamins, etc. Cell culture medium may contain extracts, e.g. serum or peptones (hydrolysates), which supply raw materials that support cell growth. Media may contain yeast-derived or soy extracts, instead of animal-derived extracts. Chemically defined medium refers to a cell culture medium in which all of the chemical components are known (i.e., have a known chemical structure). Chemically defined medium is entirely free of animal-derived components, such as serum- or animal-derived peptones. In one embodiment, the medium is a chemically defined medium.

The solution may also contain components that enhance growth and/or survival above the minimal rate, including hormones and growth factors. The solution may be formulated to a pH and salt concentration optimal for survival and proliferation of the particular cell being cultured.

A “cell line” refers to a cell or cells that are derived from a particular lineage through serial passaging or sub-culturing of cells. The term “cells” is used interchangeably with “cell population”.

The term “cell” includes any cell that is suitable for expressing a recombinant nucleic acid sequence. Cells include those of prokaryotes and eukaryotes, such as bacterial cells, mammalian cells, human cells, non-human animal cells, avian cells, insect cells, yeast cells, or cell fusions such as, for example, hybridomas or quadromas. In certain embodiments, the cell is a human, monkey, ape, hamster, rat or mouse cell. In other embodiments, the cell is selected from the following cells: Chinese Hamster Ovary (CHO) (e.g. CHO K1, DXB-11 CHO, Veggie-CHO), COS (e.g. COS-7), retinal cell, Vero, CV1, kidney (e.g. HEK293, 293 EBNA, MSR 293, MDCK, HaK, BHK21), HeLa, HepG2, WI38, MRC 5, Colo25, HB 8065, HL-60, lymphocyte, e.g. Jurkat (T lymphocyte) or Daudi (B lymphocyte), A431 (epidermal), U937, 3T3, L cell, C127 cell, SP2/0, NS-0, MMT cell, stem cell, tumor cell, and a cell line derived from an aforementioned cell. In some embodiments, the cell comprises one or more viral genes, e.g. a retinal cell that expresses a viral gene (e.g. a PER.C6® cell). In some embodiments, the cell is a CHO cell. In other embodiments, the cell is a CHO K1 cell.

### III. Systems for Characterizing Variants of Protein Drug Impurities

Multisubunit therapeutic proteins, particularly monoclonal antibody (mAb)-based therapeutics are inherently heterogeneous with respect to size due to their complex multi-chain structure and the propensity to accommodate multiple enzymatic and chemical post-translational modifications. Although the levels of size variants within a protein drug product can be readily quantitated by a variety of biophysical methods, unambiguous identification of those product-related impurities has been particularly challenging.

While mAbs possess a conserved covalent heterotetrameric structure consisting of two disulfide-linked heavy chains, each covalently linked through a disulfide bond to a light chain, these proteins often contain low levels of product-related impurities even after extensive purification steps. Low molecular weight (LMW) species (e.g., Fab fragments and monomer without an Fab arm) and high molecular weight (HMW) species (e.g., mAb trimer and mAb dimer) are both examples of product-related impurities that contribute to the size heterogeneity of mAb products. The formation of HMW species within a therapeutic mAb drug product as a result of protein aggregation can potentially compromise both drug efficacy and safety (e.g., eliciting unwanted immunogenic response) (Rosenberg A S. The AAPS journal, 8:E501-7 (2006); Moussa E M, et al. Journal of Pharmaceutical Science. 105:417-30 (2016;). LMW species of any therapeutic protein may result from host cell protease activity during production. LMW species often have low or substantially reduced activity relative to the monomeric form of the antibody, while exposing novel epitopes that can lead to immunogenicity or potentially impact pharmacokinetic properties in vivo (Vlasak J, Ionescu R. mAbs, 3:253-63 (2011)). As a result, both HMW and LMW species are considered critical quality attributes that are routinely monitored during drug development and as part of release testing of purified drug substance during manufacturing.

Molecular weight heterogeneity of mAb products is traditionally characterized by multiple orthogonal analytical methods (Michels D A, Parker M, Salas-Solano O. Electrophoresis, 33:815-26 (2012)). One of the most commonly used techniques to assess mAb product purity is SDS-PAGE, performed under non-reducing conditions. During analysis, minor bands corresponding to LMW species can be routinely observed and quantified, including H2L (2 heavy chains and 1 light chain), H2 (2 heavy chains), HL (1 heavy chain and 1 light chain), HC (1 heavy chain), and LC (1 light chain) species, with respect to antibodies (Liu H, Gaza-Bulseco G, Chumsae C, Newby-Kew A. Biotechnology Letters, 29:1611-22 (2007)).

Proteolytic fragments may also be observed. The proposed identity of each minor band can be supported by N-terminal sequencing via Edman degradation, in-gel tryptic digestion followed by mass spectrometry analysis, and western blot analysis using anti-Fc and anti-light chain antibodies.

However, any proposed structures resulting from these methods cannot be unambiguously confirmed at the intact protein level. Furthermore, sample preparation conditions employed in SDS-PAGE experiments can generate LMW artifacts through disulfide bond scrambling, which can lead to overestimations of minor LMW species (Zhu Z C, et al. Journal of Pharmaceutical and Biomedical Analysis, 83:89-95 (2013)).

More recently, capillary electrophoresis-sodium dodecyl sulfate (CE-SDS) has emerged as a modern equivalent of SDS-PAGE, offering superior reproducibility, sensitivity, and throughput (Rustandi R R, Washabaugh M W, Wang Y. Electrophoresis, 29:3612-20 (2008); Lacher N A, et al. Journal of Separation Science, 33:218-27 (2010); and Hunt G, Moorhouse K G, Chen A B. Journal of Chromatography A, 744:295-301 (1996)). During CE-SDS analysis of mAb products, minor peaks with shorter migration times (LMW forms) than the intact antibody can be routinely observed. Unlike SDS-PAGE analysis, these LMW impurities cannot be extracted or subjected to further analyses. As a result, the identities of LMW impurities observed in CE-SDS methods are often proposed solely based on empirical knowledge.

Accurate mass measurement of intact mAb proteins by modern mass spectrometers has become increasingly popular in the biopharmaceutical industry as one of the most reliable identification techniques (Kaltashov I A, et al., Journal of the American Society for Mass Spectrometry, 21:323-37 (2010)); Zhang H, Cui W, Gross M L. FEBS Letters, 588:308-17 (2014)). Specifically, a variety of “hyphenated chromatography-mass spectrometry” methods have demonstrated the capability of detecting low-abundance impurities in mAb products and providing highly detailed analyses that cannot be achieved by either SDS-PAGE or CE-SDS methods (ie J C, Bondarenko P V. Journal of the American Society for Mass Spectrometry, 16:307-11 (2005); Haberger M, et al. mAbs; 8:331-9 (2016)). For example, reversed-phase chromatography (RPLC) coupled to mass spectrometry can be used to detect free light chain and associated post-translational modifications (e.g. cysteinylation and glutathionylation) present in mAb drug products. However, compared to SDS-PAGE and CE-SDS methods, RPLC often lacks sufficient resolution to separate LMW species and thus fails to elucidate the complete LMW profile. For example, the identification of H2L species in mAb drug products has never been reported by RPLC-based intact mass analysis, owing to its low abundance and poor resolution from the main intact antibody.

Another MS-based technique that is promising for characterizing mAb product-related impurities is native electrospray ionization mass spectrometry (Native ESI-MS), which is particularly informative when coupled with size exclusion chromatography (SECX Haberger M, et al. mAbs, 8:331-9 (2016)). However, the LMW species identified in native SEC-MS analysis are often not the same as those identified by SDS-PAGE or CE-SDS, due to significantly different experimental conditions used between methods. Specifically, the sample preparation required for SDS-PAGE and CE-SDS often starts with protein denaturation, where the non-covalent interactions between the N-terminal regions of HC-LC pairs and the C-terminal regions of the HC-HC pairs are disrupted. As a result, LMW impurities such as H2L, half antibody, and free light chain species are able to dissociate from the mAb molecule if the interchain disulfide bonds are broken.

In comparison, native SEC-MS analyzes the mAb samples under near native conditions, permitting the strong non-covalent interchain interactions to be preserved and allowing the four-chain structure of the mAb molecule to be maintained even if the interchain disulfide bonds are broken. Although advances in SEC column chemistry have made it possible to use denaturing buffers (e.g. 30% acetonitrile, 0.1% FA and 0.1% TFA) that are normally used in reversed-phase chromatography for SEC separation and direct coupling to online mass spectrometry analysis (Liu H, Gaza-Bulseco G, Chumsae C. Journal of the American Society for Mass Spectrometry, 20:2258-64 (2009)), the LC resolution is still sub-optimal to detect many LMW species.

To address these challenges, a platform that couples high performance SEC and IEX separation with ultrasensitive native Nano-ESI mass spectrometry detection to allow in-depth and fast characterization of therapeutic protein drug products is provided.

A. Systems for Characterizing Size and Charge Variants in Protein Drug Products

In one embodiment the system includes a size exclusion chromatography (SEC) column, or an ion exchange chromatography (IEX) system in fluid communication with a native mass spectrometry system. The columns are suitable for use with deglycosylated proteins. In one embodiment, the SEC column is a Waters BEH® SEC column (4.6×300 mm). In one embodiment the IEX column is a strong cation exchange column. The native mass spectrometry system can be a native electrospray ionization (ESI) mass spectrometry system. In one embodiment the mass spectrometry system is a Thermo Exactive EMR mass spectrometer. The mass spectrometry system can also contain an ultraviolet light detector. The SEC and IEX columns are in fluid communication with the mass spectrometer via an analytical flow splitter that can adjust the flow rate to mass spectrometer.

In one embodiment the mobile phase is an aqueous mobile phase. A representative aqueous mobile phase contains 140 mM sodium acetate and 10 mM ammonium bicarbonate. The UV traces are typically recorded at 215 and 280 nm.

Protein drug samples are typically 5-10 ug/ul. Injection concentration is typically 50-100 ug.

In one embodiment, the size exclusion separation is achieved at room temperature, using an isocratic flow of 0.2 mL/min for 24 minutes.

In one embodiment, the voltage for electrospray is applied through the liquid junction tee right before the emitter.

B. Methods of Characterizing Protein Drug Product Impurities

The disclosed systems and methods can be used to characterize size variants, charge variants, antibody-antigen binding, PTM characterization, characterization of partially reduced and alkylated mAb, dimer characterization for co-formulated drugs, IgG4 Fab exchange characterization, and highly heterogeneous sample characterization using charge reduction. Exemplary post-translational modifications (PTMs) that can be detected and identified that contribute to acidic variants include but are not limited to glycation, glucuronylation, carboxymethylation, sialylation, non-consensus glycosylation at Fab region. PTMs that can be detected and identified that contribute to basic variants include but are not limited to succinimide formation, N-terminal glutamine (not converted to pyroglutamate), C-terminal Lys and non-/partial-glycosylated species.

1. Size Variants

One embodiment provides a method for characterizing size variants of protein drug product impurities including the steps of optionally deglycosylating a protein drug product sample, separating protein components of the protein drug product sample by native SEC chromatography using an aqueous mobile phase, and analyzing the separated protein components by mass spectrometry to characterize high molecular weight species, low molecular weight species, and intermediate high weight species of protein drug product impurities in the protein drug product sample. In one embodiment, the mobile phase includes ammonium acetate and ammonium bicarbonate.

In one embodiment the protein drug product sample is taken from or purified from a fed-batch cell culture, a continuous cell culture or a perfusion cell culture.

Exemplary protein drug products include but are not limited to an antibody, a fusion protein, recombinant protein, or a combination thereof.

Exemplary low molecular weight protein drug product impurities include but are not limited to precursors, degradation products, truncated species, proteolytic fragments including Fab, ligand or receptor fragments or heavy chain fragments, free light chain, half antibody, H2L, H2, HL, HC, or a combination thereof.

Exemplary HMW impurities include but are not limited to mAb trimers and mAb dimers.

Exemplary intermediate HMW include but are not limited to monomer with extra light chains (H2L3 and H2L4 species), monomer plus Fab fragments complexes, Fab2-Fab2, Fc-Fc, and Fab2-Fc.

2. Charge Variant Characterization

One embodiment provides a method for characterizing charge variants of protein drug product impurities including the steps of optionally deglycosylating a protein drug product sample, optionally treating the sample with IdeS from Streptocoocuspyogenes, separating protein components of the protein drug product sample by native strong cation exchange chromatography using an aqueous mobile phase, and analyzing the separated protein components by mass spectrometry to characterize charge variant species of protein drug product impurities in the protein drug product sample. In one embodiment, the mobile phase includes ammonium acetate and ammonium bicarbonate.

In one embodiment the protein drug product sample is taken from or purified from a fed-batch cell culture, a continuous cell culture or a perfusion cell culture.

Exemplary charge variants include but are not limited to glycation, glucuronylation, carboxymethylation, sialylation, non-consensus glycosylation at Fab region. PTMs that can be detected and identified that contribute to basic variants include but are not limited to succinimide formation, N-terminal glutamine (not converted to pyroglutamate), C-terminal Lys and non-/partial-glycosylated species.

C. Methods of Producing High Purity Protein Drug Products

One embodiment provides a method of producing an antibody including the steps of culturing cells producing the antibody, for example in a fed-batch culture, obtaining a sample from the cell culture, characterizing and quantifying low molecular weight, high molecular weight, and intermediate molecular weight impurities in the sample using the systems and methods disclosed herein and modifying one or more culture conditions of the cell culture to reduce the amount of characterized low molecular protein drug impurities produced during cell culture of the antibody. Typically, the conditions are changed to have the protein drug impurities in a range of 0.05% and 30.0%, preferably 0.05% to 15%, 0.05% to 10%, 0.05% to 5%, or 0.05% to 2% (w/w).

The one or more conditions of the cell culture that are changed to reduce the amount of low molecular weight protein drug impurities are selected from the group consisting of temperature, pH, cell density, amino acid concentration, osmolality, growth factor concentration, agitation, gas partial pressure, surfactants, or combinations thereof.

In one embodiment the cells producing the antibody are Chinese hamster ovary cells. In other embodiments, the cells are hybridoma cells.

Another embodiment provides an antibody produced according the methods provided herein have 1 to 5%, 5 to 10%, 10 to 15%, 15 to 20% protein drug impurities.

### EXAMPLES

### Example 1: HILIC Separation of mAb-1 Drug Substance Sample

### Methods

The SEC separation was achieved on a Waters BEH® SEC column (4.6×300 mm) that was pre-equilibrated with ammonium acetate and ammonium bicarbonate-based mobile phase at a flow rate of 0.2 mL/min. The IEX separation was achieved on a strong cation exchange column at a flow rate of 0.4 mL/min using ammonium acetate-based buffer system. An analytical flow splitter was connected after the column to reduce the flow to ˜1 μL/min prior to analysis by Thermo Exactive EMR mass spectrometer, which was equipped with a Nanospray Flex™ Ion Source. Depending on the size of the analytes, the trapping gas pressure, S-lens RF level, in-source fragmentation and HCD collision energy were adjusted to achieve optimal dissolvation.

Therefore, a new technology platform that couples high performance SEC and IEX separation with ultrasensitive native Nano-ESI mass spectrometry detection to allow in-depth and fast characterization of therapeutic mAbs is introduced.

### Results

A recombinant IgG mAb (mAb-1) drug substance sample was used as a model molecule. Utilizing SEC-MS, low levels of size variants in mAb products can be effectively separated from the main monomer species and subjected to sensitive MS detection. Both higher molecular weight species (e.g., mAb trimer and mAb dimer) and lower molecular species (e.g. Fab fragments and monomer without a Fab arm), present at <1% relative abundance, can be routinely observed and monitored by this method. In particular, an interesting category of HMW species that elute between a mAb monomer and a mAb dimer (termed as intermediate HMW species) were detected in many mAb products, even though they are typically present at extremely low levels (<0.1%). Through accurate mass measurement, the identities of those intermediate HMW species can be determined and divided into two groups: 1) monomer with extra light chains (H2L3 and H2L4 species) and 2) monomer plus Fab fragments complexes. In addition, after treatment with IdeS enzymatic digestion, different dimerized fragments (Fab2-Fab2, Fc-Fc and Fab2-Fc) can be well separated and detected by this method, revealing the dimerization interfaces at subdomain level.

Utilizing IEX-MS, a variety of PTMs contributing to charge variants can be detected at intact mAb level. Through analyses of hundreds of mAb samples, PTMs contributing to acidic variants were found to include glycation, glucuronylation, carboxymethylation, sialylation and non-consensus glycosylation at Fab region; PTMs contributing to basic variants were found to include succinimide formation, N-terminal glutamine (not converted to pyroglutamate), C-terminal Lys and non-/partial-glycosylated species. In charge variant investigations (e.g., comparability and forced degradation studies), this new approach proved to be very powerful in elucidating charge variant forms.

While in the foregoing specification this invention has been described in relation to certain embodiments thereof, and many details have been put forth for the purpose of illustration, it will be apparent to those skilled in the art that the invention is susceptible to additional embodiments and that certain of the details described herein can be varied considerably without departing from the basic principles of the invention.

All publications mentioned throughout this disclosure are incorporated herein by reference in their entirety.

All references cited herein are incorporated by reference in their entirety. The present invention may be embodied in other specific forms without departing from the spirit or essential attributes thereof and, accordingly, reference should be made to the appended claims, rather than to the foregoing specification, as indicating the scope of the invention.

We claim:

1. A protein drug product comprising:
a protein drug and an excipient, wherein the protein drug product comprises between 0.05% and 30.0% (w/w) of intermediate high molecular weight protein drug impurities.

2. The protein drug product of claim 1, wherein the protein drug product is selected from the group consisting of an antibody, a fusion protein, recombinant protein, or a combination thereof.

3. The protein drug product of claim 1, wherein the intermediate molecular weight protein drug impurities are selected from the group consisting of monomer with extra light chains including H2L3 and H2L4 species, monomer plus Fab fragments complexes, and combinations thereof.

4. The protein drug product of claim 1, wherein the drug product comprises between 0.05% to 25% w/w of intermediate high molecular weight protein drug impurities.

5. The protein drug product of claim 1, wherein the drug product comprises between 0.05% to 15% w/w of intermediate high molecular weight protein drug impurities.

6. The protein drug product of claim 1, wherein the drug product comprises between 0.05% to 10% w/w of intermediate high molecular weight protein drug impurities.

7. The protein drug product of claim 1, wherein the drug product comprises between 0.05% to 5% w/w of intermediate high molecular weight protein drug impurities.

8. A method for characterizing intermediate high molecular weight protein drug product impurities comprising:
deglycosylating a protein drug product sample;
separating protein components of the protein drug product sample by native size exclusion chromatography using an aqueous mobile phase;
analyzing the separated protein components by mass spectrometry to characterize intermediate high molecular weight protein drug product impurities in the protein drug product sample.

9. The method of claim 8, wherein the protein drug product sample is from a fed-batch culture.

10. The method of claim 8, wherein the protein drug product is selected from the group consisting of an antibody, a fusion protein, recombinant protein, or a combination thereof.

11. The method of claim 8, wherein the intermediate high molecular weight protein drug product impurity is characterized as an intermediate high molecular weight protein drug product impurity selected from the group consisting of monomer with extra light chains including H2L3 and H2L4 species, monomer plus Fab fragments complexes, and combinations thereof.

12. A method of producing an antibody, comprising:
culturing cells producing the antibody in a cell culture;
obtaining a sample from the cell culture;
characterizing and quantifying intermediate high molecular weight impurities in the sample according to the method of claim 8, and
modifying one or more culture conditions of the cell culture to reduce the amount of characterized low molecular protein drug impurities produced during cell culture of the antibody.

13. The method of claim 12, wherein the one or more conditions of the cell culture that are changed to reduce the amount of intermediate high molecular weight protein drug impurities are selected from the group consisting of pH, cell density, amino acid concentration, osmolality, growth factor concentration, agitation, gas partial pressure, surfactants, or combinations thereof.

14. The method of claim 12, wherein the cells are selected from the group consisting of bacterial cells, yeast cells, Chinese Hamster Ovary (CHO) cells (e.g. CHO K1, DXB-11 CHO, Veggie-CHO), COS cells (e.g. COS-7), retinal cells, Vero cells, CV1 cells, kidney cells (e.g. HEK293, 293 EBNA, MSR 293, MDCK, HaK, BHK21), HeLa cells, HepG2 cells, WI38 cells, MRC 5 cells, Colo25 cells, HB 8065 cells, HL-60 cells, lymphocyte cells, e.g. autologous T cells, Jurkat (T lymphocytes) or Daudi (B lymphocytes), A431 (epidermal) cells, U937 cells, 3T3 cells, L cells, C127 cells, SP2/0 cells, NS-0 cells, MMT cells, stem cells, tumor cells, and a cell line derived from any of the aforementioned cells.

15. The method of claim 12, wherein the cells are hybridoma cells or quadroma cells.

16. The antibody produced by the method of claim 12.

17. The antibody of claim 16, comprising 0.05 and 30.0% (w/w) of intermediate high molecular weight protein drug impurities.

18. A system for characterizing intermediate high molecular weight drug impurities, comprising:
a native size exclusion chromatography system comprising a size exclusion column linked to a mobile phase column comprising an aqueous mobile phase, wherein the size exclusion column is in fluid communication with a Nano-ESI mass spectrometry system.

19. A method for characterizing charge variant drug impurities, comprising:
deglycosylating a protein drug product sample;
separating protein components of the protein drug product sample by native strong cation exclusion chromatography using an aqueous mobile phase;
analyzing the separated protein components by Nano-ESI mass spectrometry to characterize charge variant protein drug product impurities in the protein drug product sample.

20. The method of claim 19, wherein the protein drug product sample is from a fed-batch culture.

21. The method of claim 19, wherein the protein drug product is selected from the group consisting of an antibody, a fusion protein, recombinant protein, or a combination thereof.

22. The method of claim 19, wherein the intermediate high molecular weight protein drug product impurity is characterized as an intermediate high molecular weight protein drug product impurity selected from the group consisting of monomer with extra light chains including H2L3 and H2L4 species, monomer plus Fab fragments complexes, and combinations thereof.

23. The method of claim 8, wherein the aqueous mobile phase comprises ammonium acetate and ammonium bicarbonate.
